(19) United States
(12) Patent Application Publication
(10) Pub. No.: US 2008/0233216 A1
(43) Pub. Date: Sep. 25, 2008

(54) Composition Comprising the Extract of Siegesbeckiae Herba For Preventing and Treating Arthritis and the Use Thereof

(75) Inventors: Dong-Suk Park, Seoul (KR); Myung Chul Yoo, Seoul (KR); Do-Young Choi, Gyeonggi-do (KR); Jae-Dong Lee, Seoul (KR); Yong-Baik Cho, Gyeonggi-do (KR); Nam-Jae Kim, Seoul (KR); Eun-Mi Cho, Seoul (KR); Jeong-Eun Huh, Seoul (KR); Yong-Hyeon Baek, Seoul (KR); Seung Jae Hong, Seoul (KR)

Correspondence Address: KIRK HAHN, 14431 HOLT AVE, SANTA ANA, CA, 92705, US

(73) Assignee: Industry Academic Cooperation Foundation Of Kyunghee University, Gyeonggi-do (KR)

(21) Appl. No.: 12/064,449

(22) PCT Filed: Feb. 24, 2006
(86) PCT No.: PCT/KR2006/000647

§ 371 Date: Feb. 21, 2008

(30) Foreign Application Priority Data
Jan. 19, 2006 (KR) 10-2006-0005684

(51) Int. Cl. A61K 36/00; A61P 19/02

(52) U.S. Cl. 424/725

(57) ABSTRACT

The extract of Siegesbeckiae herba of the present invention showed potent inhibitory effect on the dissociation of proteoglycan and type II collagen in chondrocyte and cartilage tissue and protecting effect on cartilage due to the inhibition of MMP-1, MMP-3 and MMP-13 activity and the restoring effect on cartilage tissue, the anti-inflammatory and antiphlogistic effect in edema animal model, anti-inflammatory effect confirmed by the inhibition test on PGE2 activity through COX-2 inhibition and the inhibition test of the reproduction of TNF-α and NO, it can be used as the therapeutics or health food for treating and preventing arthritic disease.

### TECHNICAL FIELD

The present invention relates to composition comprising the extract of Siegesbeckiae herba for preventing and treating arthritis and the use thereof.

### BACKGROUND ART

Degenerative arthritis, one of representative osteo-joint diseases is chronic arthritis. It is difficult to treat the disease with conventionally available anti-inflammatory drugs in clinic. Moreover, the drugs give rise to systemic adverse response such as digestive disorder, gastro-intestinal disorder and renal function disorder and the adverse response of the drugs occurs more frequently as the age of patient increases, which causes to lots of problems in case of long-term systemic treatment in older people. Therefore, the new drug development targeting anti-inflammatory effect, protecting and regenerating effect on cartilage has been urgently needed than previous systemic treatment therapy recently. The recent way of drug development has been focused on joint tissue lyase inhibitor, free radical scavenger such as SOD, conservation therapy using by long-term treatment of joint tissue components such as chondroitin or glucosamine etc (Badger A. M. et al., J. Pharmacol. Exp. Ther., 290, pp 587-593, 1999; Choi J. H. et al., Osteoarthritis Cartilage, 10(6) pp 471-478, 2002).

Various biochemical mechanisms, in particular, nitric oxide synthase (NOS) enzyme generating nitric oxide and the other enzymes involve in the synthesis of prostaglandin (PGs) play an important role in the etiological factor of arthritis in vivo. Accordingly, NOS enzyme generating NO from L-Arginine or cyclooxygenase (COX) involving in the synthesis of various prostaglandins have been main target to block the inflammation of arthritis.

According to recent report, there are several kinds of NOS enzymes, for example, bNOS (brain NOS) existing in brain, nNOS (neuronal NOS) in neuronal system, eNOS (endothelial NOS) in endothelial system etc, which are expressed at regular level in human body. A little NO reproduced thereby plays an important role in maintaining of homeostasis such as neuronal transmission or induction of vasodilation etc whereas excess amount of NO occurring abruptly by iNOS (induced NOS) induced by various cytokines or external stimulator gives rise to cell toxicity or inflammatory reaction. Chronic inflammation is correlated with the increased activity of iNOS (Chan P. S. et al., Osteoarthritis cartilage, 13(5), pp 387-394, 2005; Appleton I. et al., Adv. Pharmacol., 35, pp 27-28, 1996).

There exist two iso-types of cyclooxygenase, i.e., cyclooxygenase-1 (COX-1) being present in the cell all the time and showing synthesizing activity of PGs necessary in cell protection and cyclooxygenase-2 (COX-2) being abruptly increased in cell and playing an important role in inflammatory reaction (Weisz A et al., Biochem. J., 316(Pt1), pp 209-215, 1996). The transcription inflammatory factor including iNOS and COX-2 increasing the rate of NO and PGs is correlated with the etiology of chronic disease.

Siegesbeckiae herba is an aerial part of Siegesbeckia spp. belonged to Compositae. There are several plants belonged to Siegesbeckia spp., for example, Siegesbeckia glabrescens, S. pubescens, S. orientalis or Siegesbeckia glabrescens which usually grow on roadside and steep slope of the mountain. They have been conventionally used to treat or prevent several diseases such as diplegia, myalgia, acute hepatitis, hypertension, hemorrhage of traumatic injury etc and they have been known to contain diterpenes such as 16-acetyl-kirenol, darutigenol, darutoside, isopropylidenekirenol, kirenol, neodarutoside, siegesbeckiol, siegesbeckioside etc as main components ( [symbol] JungyakDaesacheon, Jungdamsa, pp 73-79, 1997).

There have several reports on the medicinal activity of Siegesbeckiae herba for, example, inhibitory effect on histamine release, stimulating activity of hair growth etc (Kang B. K. et al., J. Ethnopharmacol., 57(2), pp 73-79, 1997; Korean Patent No. 165937), however, there has been not reported or disclosed about the therapeutic effect of Siegesbeckiae herba on the arthritic disease in any of above cited literatures, the disclosures of which are incorporated herein by reference.

Accordingly, the present inventors have confirmed that the extract of Siegesbeckiae herba shows potent anti-inflammatory effect through various experiments, i.e., the inhibitory effect on the dissociation of proteoglycan and type II collagen in chondrocyte and cartilage tissue and protecting effect on cartilage due to the inhibition of MMP-1, MMP-3 and MMP-13 activity and the restoring effect on cartilage tissue, the anti-inflammatory and antiphlogistic effect in edema animal model, anti-inflammatory effect confirmed by the inhibition test on PGE2 activity through COX-2 inhibition and the inhibition test of the reproduction of TNF-α and NO, therefore, it can be used as the effective and safe therapeutics or health food for treating and preventing arthritic disease.

### DISCLOSURE OF INVENTION

### Technical Problem

Accordingly, it is an object of the present invention to provide a pharmaceutical composition comprising the extract of Siegesbeckiae Herba as an active ingredient for the treatment and prevention of arthritic diseases by way of stimulating the recovery of cartilage tissue, protecting cartilage damage due to the stimulation of cartilage component and inhibition of cartilage dissociation, and inhibiting inflammation and pain and the use thereof.

### Technical Solution

It is an object of the present invention to provide a pharmaceutical composition comprising the extract of Siegesbeckiae Herba as an active ingredient for the treatment and prevention of arthritic diseases by way of stimulating the recovery of cartilage tissue, protecting cartilage damage due to the stimulation of cartilage component and inhibition of cartilage dissociation, and inhibiting inflammation and pain and the use thereof.

The present invention provides a pharmaceutical composition comprising the extract of Siegesbeckiae Herba for preventing and treating of arthritic disease.

The term “extract” disclosed herein includes the extract soluble in water, C to C lower alcohol and the mixture thereof, preferably, the mixture of water and ethanol.

The term “arthritic disease” disclosed herein includes degenerative arthritis and rheumatic arthritis.

The present invention also provided a use of an extract of Siegesbeckiae Herba for the preparation of therapeutic agent for the treatment and prevention of arthritic disease in mammal or human.

The present invention also provided a pharmaceutical composition comprising an extract of Siegesbeckiae Herba and a pharmaceutically acceptable carrier thereof as an active ingredient for treating and preventing arthritic disease.

It is an object of the present invention to provide a method of treating or preventing arthritic disease in mammal or human comprising administering to said mammal or human with an effective amount of an extract of Siegesbeckiae Herba, together with a pharmaceutically acceptable carrier thereof.

Hereinafter, the present invention is described in detail.

An inventive extract of Siegesbeckiae Herba can be prepared in detail by following procedures.

For example, Siegesbeckiae Herba is dried, cut, crushed and mixed with 1 to 20-fold, preferably, approximately 5 to 15-fold volume of distilled water, C₁ to C₄ lower alcohols or the mixtures thereof, preferably the mixture of water and ethanol with approximately 1:0.1 to 1:10, more preferably, 1:0.2 to 1:5 with mixing ratio (kg/L); the solution is treated with hot water at the room temperature, preferably, for the period ranging from 1 to 10 hours with the extraction method by the extraction with hot water, cold water, reflux extraction, or ultra-sonication extraction, preferably, extraction with hot water; the extract is collected with filtration, concentrated under reduced pressure and dried to obtain an inventive extract of Siegesbeckiae Herba.

Also, the above-described procedures may be modified or subjected to further step to fractionate or isolate more potent fractions or compounds by conventional procedure well-known in the art, for example, the procedure disclosed in the literature (Harborne J. B. Phytochemical methods: A guide to modern techniques of plant analysis, 3_{rd} Ed. pp 6-7, 1998).

It is confirmed that the extract of Siegesbeckiae herba prepared by above-described method shows potent anti-inflammatory effect through various experiments, i.e., the inhibitory effect on the dissociation of proteoglycan and type II collagen in chondrocyte and cartilage tissue and protecting effect on cartilage due to the inhibition of MMP-1, MMP-3 and MMP-13 activity and the restoring effect on cartilage tissue, the anti-inflammatory and antiphlogistic effect in edema animal model, anti-inflammatory effect confirmed by the inhibition test on PGE2 activity through COX-2 inhibition and the inhibition test of the reproduction of TNF-α and NO.

The present invention, there are provided a pharmaceutical composition comprising an extract of Siegesbeckiae Herba and a pharmaceutically acceptable carrier thereof as an active ingredient for treating and preventing arthritic diseases.

The inventive composition for treating and preventing arthritic diseases may comprises the above-described extract as 0.1˜50% by weight based on the total weight of the composition.

The inventive composition may additionally comprise conventional carrier, adjuvants or diluents in accordance with a using method well known in the art. It is preferable that said carrier is used as appropriate substance according to the usage and application method, but it is not limited. Appropriate diluents are listed in the written text of Remington's Pharmaceutical Science (Mack Publishing co, Easton Pa.).

Hereinafter, the following formulation methods and excipients are merely exemplary and in no way limit the invention.

The composition according to the present invention can be provided as a pharmaceutical composition containing pharmaceutically acceptable carriers, adjuvants or diluents, e.g., lactose, dextrose, sucrose, sorbitol, mannitol, xylitol, erythritol, maltitol, starches, acacia rubber, alginate, gelatin, calcium phosphate, calcium silicate, cellulose, methyl cellulose, polyvinyl pyrrolidone, water, methylhydroxy benzoate, propylhydroxy benzoate, talc, magnesium stearate and mineral oil. The formulations may additionally include fillers, anti-agglutinating agents, lubricating agents, wetting agents, flavoring agents, emulsifiers, preservatives and the like. The compositions of the invention may be formulated so as to provide quick, sustained or delayed release of the active ingredient after their administration to a patient by employing any of the procedures well known in the art.

For example, the compositions of the present invention can be dissolved in oils, propylene glycol or other solvents that are commonly used to produce an injection. Suitable examples of the carriers include physiological saline, polyethylene glycol, ethanol, vegetable oils, isopropyl myristate, etc., but are not limited to them. For topical administration, the extract of the present invention can be formulated in the form of ointments and creams.

Pharmaceutical formulations containing present composition may be prepared in any form, such as oral dosage form (powder, tablet, capsule, soft capsule, aqueous medicine, syrup, elixirs pill, powder, sachet, granule), or topical preparation (cream, ointment, lotion, gel, balm, patch, paste, spray solution, aerosol and the like), or injectable preparation (solution, suspension, emulsion).

The composition of the present invention in pharmaceutical dosage forms may be used in the form of their pharmaceutically acceptable salts, and also may be used alone or in appropriate association, as well as in combination with other pharmaceutically active compounds.

The desirable dose of the inventive extract or composition varies depending on the condition and the weight of the subject, severity, drug form, route and period of administration, and may be chosen by those skilled in the art. However, in order to obtain desirable effects, it is generally recommended to administer at the amount ranging 0.1 to 1000 mg/kg, preferably, 1 to 100 mg/kg by weight/day of the inventive extract of the present invention. The dose may be administered in single or divided into several times per day. In terms of composition, the amount of inventive extract should be present between 0.01 to 50% by weight, preferably 0.5 to 40% by weight based on the total weight of the composition.

The pharmaceutical composition of present invention can be administered to a subject animal such as mammals (rat, mouse, domestic animals or human) via various routes. All modes of administration are contemplated, for example, administration can be made orally, rectally or by intravenous, intramuscular, subcutaneous, intracutaneous, intrathecal, epidural or intra-cerebroventricular injection.

Also, it is another object of the present invention to provide a health food or food additives comprising an extract of Siegesbeckiae herba, together with a sitologically acceptable additive for the prevention and alleviation of arthritic diseases. The health food of the present invention comprises the above-described extract as 0.01 to 80%, preferably 1 to 50% by weight based on the total weight of the composition.

The health food of the present invention can be contained in health food, health beverage etc, and may be used as powder, granule, tablet, chewing tablet, capsule, beverage etc.

The health food of the present invention comprises the above-described extract as 0.01 to 80%, preferably 1 to 50% by weight based on the total weight of the composition.

The food additive of the present invention can be contained in health food, health beverage etc, and may be used as powder, granule, tablet, chewing tablet, capsule, beverage etc.

Also, the present invention provide a composition of the health food beverage for the prevention and improvement of arthritic diseases adding 0.01 to 80% the above-described extract by weight, 0.001 to 5% amino acids by weight, 0.001 to 2% vitamins by weight, 0.001 to 20% sugars by weight, 0.001 to 10% organic acids by weight and proper amount of sweetener and flavors.

To develop for health food, examples of addable food comprising the above-described extract of the present invention are various food, beverage, gum, vitamin complex, health improving food and the like, and can be used as power, granule, tablet, chewing tablet, capsule or beverage etc.

Also, the extract of the present invention will be able to prevent and alleviate arthritic disease by way of adding to child and infant food, such as modified milk powder, modified milk powder for growth period, modified food for growth period.

The above-described composition therein can be added to food, additive or beverage, wherein, the amount of above described extract in food or beverage may generally range from about 0.1 to 80 w/w %, preferably 1 to 50 w/w % of total weight of food for the health food composition and 1 to 30 g, preferably 3 to 10 g on the ratio of 100□ of the health beverage composition.

Providing that the health beverage composition of present invention contains above described extract as an essential component in the indicated ratio, there is no particular limitation on the other liquid component, wherein the other component can be various deodorant or natural carbohydrate etc such as conventional beverage. Examples of aforementioned natural carbohydrate are monosaccharide such as glucose, fructose etc; disaccharide such as maltose, sucrose etc; conventional sugar such as dextrin, cyclodextrin; and sugar alcohol such as xylitol, and erythritol etc. As the other deodorant than aforementioned ones, natural deodorant such as taumatin, stevia extract such as levaudioside A, glycyrrhizin et al., and synthetic deodorant such as saccharin, aspartam et al., may be useful favorably. The amount of above described natural carbohydrate is generally ranges from about 1 to 20 g, preferably 5 to 12 g in the ratio of 100□ of present beverage composition.

The other components than aforementioned composition are various nutrients, a vitamin, a mineral or an electrolyte, synthetic flavoring agent, a coloring agent and improving agent in case of cheese chocolate et al., pectic acid and the salt thereof, alginic acid and the salt thereof, organic acid, protective colloidal adhesive, pH controlling agent, stabilizer, a preservative, glycerin, alcohol, carbonizing agent used in carbonate beverage et al. The other component than aforementioned ones may be fruit juice for preparing natural fruit juice, fruit juice beverage and vegetable beverage, wherein the component can be used independently or in combination. The ratio of the components is not so important but is generally range from about 0 to 20 w/w % per 100 w/w % present composition. Examples of addable food comprising aforementioned extract therein are various food, beverage, gum, vitamin complex, health improving food and the like.

The inventive composition may additionally comprise one or more than one of organic acid, such as citric acid, fumaric acid, adipic acid, lactic acid, malic acid; phosphate, such as phosphate, sodium phosphate, potassium phosphate, acid pyrophosphate, polyphosphate; natural anti-oxidants, such as polyphenol, catechin, α-tocopherol, rosemary extract, vitamin C, green tea extract, licorice root extract, chitosan, tannic acid, phytic acid etc.

### ADVANTAGEOUS EFFECTS

The extract of siegesbeckiae herba of the present invention shows potent anti-inflammatory effect through various experiments, i.e., the inhibitory effect on the dissociation of proteoglycan and type II collagen in chondrocyte and cartilage tissue and protecting effect on cartilage due to the inhibition of MMP-1, MMP-3 and MMP-13 activity and the restoring effect on cartilage tissue, the anti-inflammatory and antiphlogistic effect in edema animal model, anti-inflammatory effect confirmed by the inhibition test on PGE activity through COX-2 inhibition and the inhibition test of the reproduction of TNF-α and NO, therefore it can be used as the effective and safe therapeutics or health food for treating and preventing arthritic diseases.

### BRIEF DESCRIPTION OF THE DRAWINGS

The above and other objects, features and other advantages of the present invention will more clearly understood from the following detailed description taken in conjunction with the accompanying drawings, in which;

FIG. 1 shows the concentration of GAG (Glycosaminoglycan) in culture medium when the inflamed cartilage tissue of human and rabbit induced by interlukin-1 (IL-1) was cultured after the extract of Siegesbeckiae herba had been treated thereto;

FIG. 2 shows the expression of proteoglycan gene using by extracted RNA chondrocyte Siegesbeckiae herba had been treated thereto;

FIG. 3 presents the concentration of type II Collagen in culture medium when the inflamed cartilage tissue of human and rabbit induced by interlukin-1 (IL-1) was cultured after the extract of Siegesbeckiae herba had been treated thereto;

FIG. 4 presents the expression of type II Collagen gene by using RNA when the inflamed cartilage tissue and chondrocyte of human and rabbit induced by interlukin-1 (IL-1) was cultured after the extract of Siegesbeckiae herba had been treated thereto;

FIG. 5. represents the concentration of MMP-1 and MMP-13 in culture medium using by ELISA method when the inflamed cartilage tissue and chondrocyte of human and rabbit induced by interlukin (IL-1) was cultured after the extract of Siegesbeckiae herba had been treated thereto;

FIG. 6 represents the expression of MMP-1, MMP-3 and MMP-13 gene in culture medium using by extracted RNA when the inflamed cartilage tissue and chondrocyte of human and rabbit induced by interlukin-1 (IL-1) was cultured after the extract of Siegesbeckiae herba had been treated thereto;

FIG. 7. depicts the cell toxicity according to the treatment of the extract of Siegesbeckiae herba when the inflamed cartilage tissue of human and the chondrocyte of rabbit with induced by interlukin-1 (IL-1) was cultured after the extract of Siegesbeckiae herba had been treated thereto;

FIG. 8. depicts the morphological change of the inflamed cartilage tissue of human and the chondrocyte of rabbit with induced by interlukin-1 (IL-1) stained with safranin O and trichrome when the inflamed cartilage tissue of human and the chondrocyte of rabbit with induced by interlukin-1 (IL-1) was cultured after the extract of Siegesbeckiae herba had been treated thereto;

### BEST MODE FOR CARRYING OUT THE INVENTION

The present invention is more specifically explained by the following examples. However, it should be understood that the present invention is not limited to these examples in any manner.

### Mode for the Invention

### Example 1

### Preparation of the Extract of Siegesbeckiae Herba

250 g of Siegesbeckiae herba procured from Kyunghee medical center located in Seoul was cut into small pieces with the size of about 1.0 cm, mixed with 2 liter of 50% ethanol thoroughly and the mixture was subjected to reflux extraction for 6 hours.

The solution was filtered with filter paper and the filtrate was collected. The remaining residue was collected and 1.5 liter of 30% (v/v) ethanol aqueous solution was added thereto to extract again for 3 hours. The collected residue was concentrated, dried with lyophilization and then obtained 31.1 g of a powder of Siegesbeckiae herba to use as a test sample in following experiments (designated as ‘SP’ hereinafter).

### Reference Example 1

### Preparation of Cartilage Cell and Cartilage Tissue

The jointcartilage sample of human was provided from the patient taken artificial joint surgery (Orthopedics Surgery Dep. of Kyunghee Medical Center) and the joint cartilage of rabbit was collected from 5-weeks old rabbit (New Zealand White Rabbit, Samtako Biokorea Co., Korea). The chondrocyte sample was separated from 2-weeks old rabbit (New Zealand White Rabbit, Samtako Biokorea Co., Korea).

1-1. Culture of Cartilage Tissue

After revealing the surface of joint by surgery under sterilized condition, about 200-220 mg of articular surface tissue prepared from the articular cartilage of human and rabbit was dipped in DMEM medium (FBS, GIBCO BRL, USA) supplemented with 5% fetal bovine serum and 100 unit/ml of penicillin-streptomycin. The tissue was washed with the above medium several times and then the articular tissue was cultured at 37° C. in humidified 5% CO incubator. 1 or 2 days after the incubation, the above medium was replaced with new basic medium containing inactivated 5% fetal bovine serum with heat treatment, 10 mM HEPES, and 100 unit/ml of penicillin-streptomycin, and 30 mg of the articular tissue was transferred to 48-well plate. 0.01, 0.2, and 0.4 mg/ml of the extract of Siegesbeckiae herba were treated thereto.

After culturing for 1 hours, 5 ng/ [symbol] of interlukin-1α (IL-1α, R&D system, USA) was added to the medium to induce inflammation and the medium was further cultured at 37° C. After culturing for 3 days, the medium was collected and replaced with fresh medium containing an extract of Siegesbeckiae herba and 50 μM diclofenac. The medium was further cultured for 25 days and each culture solution was collected at the 3^{rd}, 7^{th}, 14^{th}, 28^{th} day. The solutions were stored −20° C. samples.

1-2. Culture of Cartilage Cell

An articular tissue was dipped into the culture dish filled with phosphate buffered solution (PBS) under sterilized condition and the unessential flesh of articular tissue was removed by scissor to isolate bone. The sliced cartilage tissues having the thickness of about 0.5 mm and the size of 2×2 mm were washed three times with PBS and 0.2% collagenase-2 (Sigma, USA) was added thereto. The medium was stirred at the interval of 30 minutes in CO_{z} incubator for 6 hrs. After centrifuging at the speed of 1200 rpm for 5 minutes, the supernatant was collected in new tube and the procedure was repeated three times to collect purified cartilage cells.

After distributing the cartilage cells to the concentration of 28.36×10⁵ cells/100 mm² per each culture dish and the medium was sub-cultured under confluent condition. After distributing the cartilage cell to the concentration of 1.0×10⁵ cell/ [symbol] in 6 well plates and 96 well plates when the cells reached to the 5^{th} generation, 0.1, 1, 10 [symbol] of the extract of Siegesbeckiae herba were added to 6-wells and 96-wells and incubated for 48, 72 hours respectively. 5 ng/ [symbol] of interlukin-1 (R&D system, USA) was added to the well plates to induce inflammation and incubated at 37° C. for 3 days.

The culture medium was recovered and the medium was transferred to fresh medium added with the extract of Siegesbeckiae herba to incubate for 25 days. After adding the samples, the supernatant of culture medium was collected at the 3^{rd}, 7^{th}, 14^{th} and 28^{th} day and stored at −20° C.

### Reference Example 2

### Reverse Transcription Polymerase Chain Reaction (RT-PCR)

The cartilage cell incubated with the method disclosed in Reference Example 1-2 was treated with TRIzol reagent (Invitrogen Corporation, CA, USA) to isolate RNA and reverse transcription for 1 [symbol] of total RNA was performed by adding buffer solution containing oligo(dT)₁₂ primer, Dntp (10 mM), 0.1 M dithiothreitol (DDT), reverse transcriptase and RNase inhibitor to the medium and incubating the medium at 42° C. for 60 minutes. PCR (polymerase Chain Reaction) using by the primers disclosed in Table 1 and Sequence ID NOs. 1 to 12 was performed by using 1 [symbol] of each cDNA synthesized in the above, 2.5 unit of Taq polymerase enzyme (TaKaRa Taq™, Takara, Japan), 1.5 mM dNTP, 1× buffer solution (10 mM Tris-HCl pH 8.3, 50 mM KCl, Triton X-100), and 20 pM of each paired primers in Table 1 and Sequence ID NOs. 1 to 12 and the solution was adjusted with distilled water to total volume of 10 [symbol] The PCR was performed using by thermal cycler apparatus (Bio-Rad, USA) according to following procedure. After denaturing at 94° C. for 5 minutes, the PCR is performed in the order of reaction for 60 sec at 94° C., 60 sec at 55° C., 90 sec at 72° C. The cycles were repeated 30 times and the last extension was performed at 72° C. for 5 minutes. The product produced by PCR was subjected to electrophoresis (5 V/cm) in 1.8% agarose gel and stained for 5 minutes with 2 [symbol] of ethidium bromide (EtBr). The stained product was washed for 10 minutes with distilled water and the result was determined at UV wavelength (260 nm).

TABLE 1

| | |
|---|---|
| Gene | Primer Sequence |

| | | |
|---|---|---|
| Col II | Sense | AAC ACT GCC AAC GTC CAG AT |
| | Anti-sense | CTG CAG CAC GGT ATA GGT GA |
| PG | Sense | GAG GTC GTG GTG AAA GGT GT |
| | Anti-sense | GTG TGG ATG GGG TAC CTG AC |
| MMP-1 | Sense | AAA GGG AAT AAG TAC TGG G |
| | Anti-sense | GTT TTT CCA GTG TTT TCC TCA G |
| MMP-3 | Sense | TGC GTG GCA GTT TGC TCA GCC |
| | Anti-sense | GAA TGT GAG TGG AGT CAC CTC |
| MMP-13 | Sense | GAT AAA GAC TAT CCG AGA C |
| | Anti-sense | CGA ACA ATA CGG TTA CTC |
| GAPDH | Sense | GCT CTC CAG AAC ATC ATC CCT GCC |
| | Anti-sense | CGT TGT CAT ACC AGG AAA TGA GCT |

### Experimental Example 1

### Protective Effect on Cartilage

Generally, the cartilage is worsen and arthritis occurs caused by late production rate of proteoglycan or collagen in cartilage, which results in loss of cushion function. The articular cartilage consists of water (70˜80%) necessary for lubrication and growth, collagen (10˜15%), proteoglycan (5˜10%) and chondrocyte, wherein proteoglycan has particular structure with core protein attached with several glycosaminoglycan (GAG).

In order to confirm the inhibitory effect of an inventive extract on proteoglycan and collagen dissociation, following experiment was performed in the procedure.

1-1. Effect on Dissociation of Glycosaminoglycan

In order to determine the protective effect on the articular cartilage tissue of rabbit and human, 1,9-dimethylmethylene blue (DMB) assay method was performed by the procedure disclosed in the literature to confirm the inhibitory effect on the dissociation of GAG consisting of proteglycan (French M M et al., Ann. Biomed. Eng., 32(1) pp 50-56, 2004).

The concentration of GAG in the culture medium of cartilage tissue incubated with the procedure disclosed in Reference Example 1-1 was measured by determining the amount of polyanionic substance produced by being reacted with blyscan dye solution and chondroitin sulfate was used as a standard. The mixture mixed with 50 [symbol] of culture medium treated with the extract of Siegesbeckiae herba and 50 [symbol] of the culture medium treated with diclofenac (Sigma, USA) as a positive control, was mixed with 500 [symbol] of blyscan dye solution and reacted for 30 minutes at room temperature. The reactant was centrifuged at 12,000 rpm for 10 minutes and the precipitate was dissolved in blyscan dye dissociation solution. The amount of spectroscopic GAG was determined at 540 nm and the inhibition rate was expressed based on the amount of dissociated GAG induced by interlukin-1α(IL-1α).

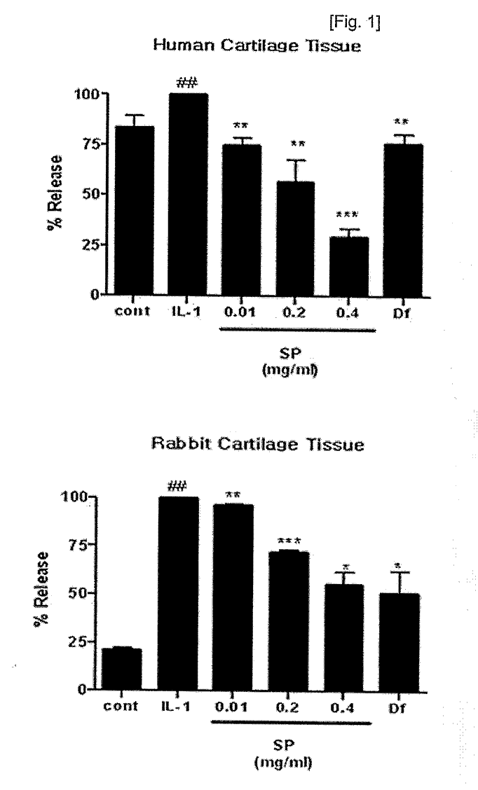

As shown in FIG. 1, the inventive extract of the present invention potently inhibited the dissociation of GAG to the medium, which confirms that the inventive extract of the present invention inhibited the dissociation of proteoglycan in cartilage induced by IL-1α in a dose dependent manner, and furthermore, it more inhibited the dissociation of GAG in human cartilage tissue comparing with diclofenac used as a positive control.

1-2. Gene Expression of Proteoglycan Gene

The expression of proteoglycan gene collected from the cartilage tissue and chondrocytic cell of rabbit in above Reference Example 1-1 and 1-2 was determined by using the primers of Sequence ID NOs. 3, 4, 11 and 12 and the test was performed by using reverse transcription polymerase chain reaction (RT-PCR) with the method disclosed in Reference Example 2.

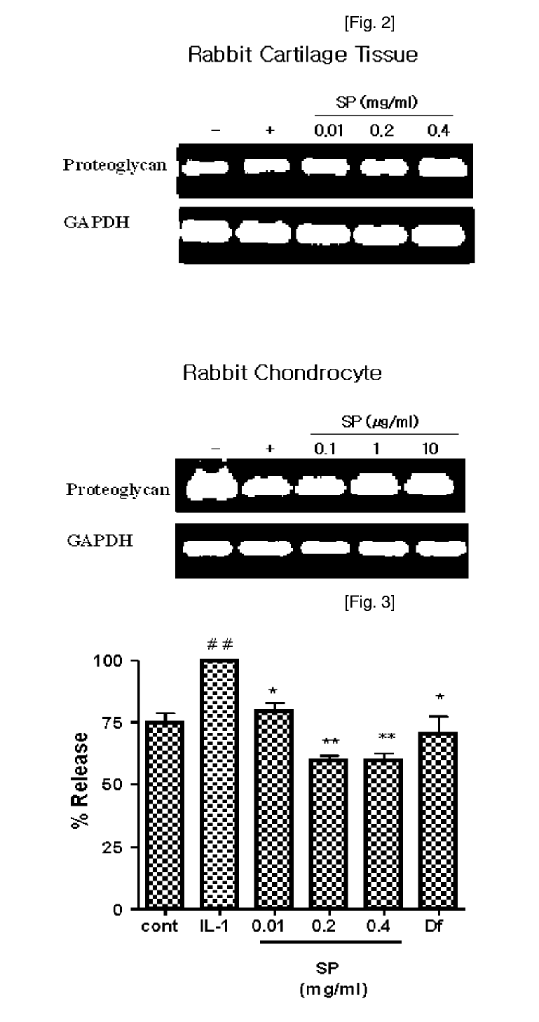

As shown in FIG. 2, the inventive extract of the present invention potently inhibited the gene expression of proteoglycan inhibited by the treatment of IL-1α in cartilage tissue and chondrocyte in rabbits in a dose dependent manner.

1-3. Determination of the Concentration of Type II Collagen in Medium

In order to determine the protective effect of the inventive extract on cartilage cell, the inhibitory effect on the collagen dissociation in articular cartilage has been tested in accordance with following procedure:

The level of type II collagen (Col II) in the medium was determined by Sircol collagen assay method disclosed in the literature (Liu X D et al., J. Lab. Clin. Med. 137(3), pp 208-219, 2001).

The sample was reacted with Sirius red dye including sulfonic acid at room temperature for 30 minutes and the optical density of reactant solution was determined at 540 nm. The inhibition rate (%) of Col II dissociation was calculated based on the amount of dissociated Col II induced by IL-α in articular tissue.

As shown in FIG. 3, the inventive extract potently inhibited the concentration of Col II. Therefore it has confirmed that it recovered the collagen production in injured cartilage tissue. Comparing with the effect of diclofenac used as a positive control, the inventive extract more potently inhibited the collagen dissociation induced by IL-α at the concentration of 0.2 mg/ml.

1-4. Determination of Col II Gene Expression

The Col II gene expression of the cartilage tissue and chondrocyte in rabbit prepared in Reference Example 1-1 and 1-2 was subjected to RT-PCR using by the primers of Sequence ID NOs. 1, 2, 11, 12 according to the procedure disclosed in Reference Example 2.

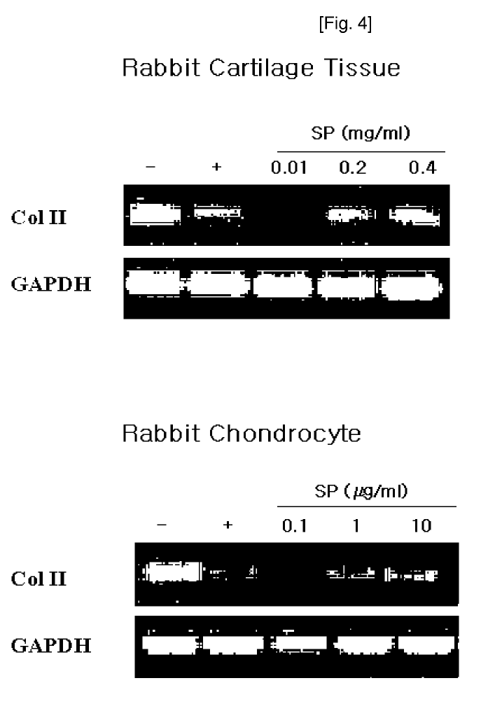

As shown in FIG. 4, the inventive extract of the present invention significantly inhibited the Col II gene expression at the concentration of 0.01 mg/ml, which confirmed that the inventive extract inhibited the dissociation of collagen.

1-5. Determination of the Concentrations of MMP-1 and MMP-13 in Medium

Matrix metalloproteinase (MMP), a protease cleaving the protein in cartilage tissue, destroys the cartilage tissue in rheumatic arthritis and osteoarthritis resulting in exacerbating arthritis. Accordingly, the inhibition of the enzyme production is main target to protect articular articular cartilage (Nagase H and Woessner J F Jr., J. Biol. Chem., 274(31), pp 21491-21494, 1999).

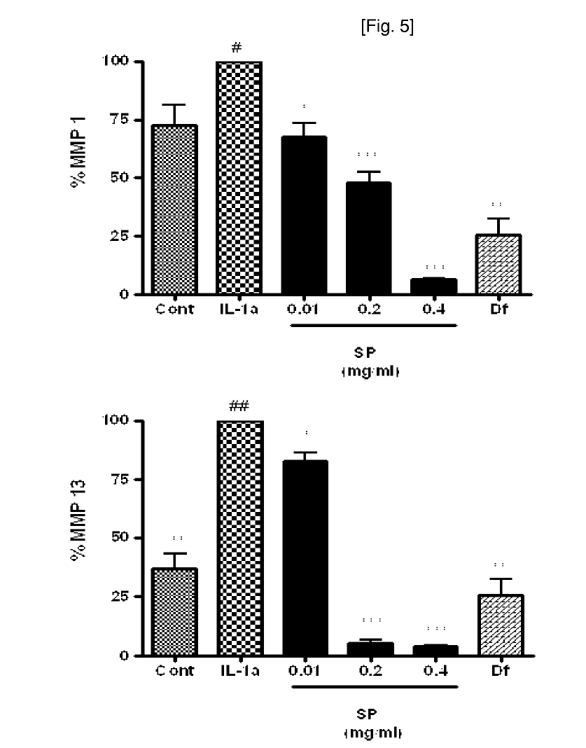

The inhibitory effect on MMP reproduction using human cartilage tissue medium prepared in Reference Example 1-1 was determined by using ELISA kit (MMP-1 kit, MMP-13 kit, Biomol Research Lab., Inc., PA, USA), according to the manual of manufacture and thiopeptolide (Ac-Prop Leu-Gly-[2-mercapto-4-methyl-pentanoyl]-Leu-Gly-OC₂H₅) was used as a colorimetric substrate excised by MMP-1 (collagenase-1) and MMP-13 (collagenase-13). In order to measure proteolytic activity, each 25 [symbol] of medium was added to 96-well plate with 50 [symbol] of the substrate to incubate at 37° C. for 1 hr and the optical density was measured by ELISA reader (Molecular devices, USA) at 450 nm. The activity of each sample on MMP-1 and MMP-13 was determined by calculating the MMP (%) of medium in each well and the result was shown in FIG. 5.

The inventive extract of the present invention significantly inhibited the activity of collagenase MMP-1 and MMP-13 in a dose dependent manner. Comparing with the effect of diclofenac (Df) used as a positive control, the inventive extract more potently inhibited the activity of collagenase MMP-1 and MMP-13 at the concentration of 0.2 mg/ml.

1-6. Inhibition of the Gene Expression of MMP-1, MMP-3 and MMP-13

To determine the inhibitory effect of inventive extract on the reproduction of MMPs (Matrix Metalloproteinases) using the cartilage tissue and chondrocyte of rabbit prepared in Reference Example 1-1 and reference 1-2, RT-PCR was performed by using the primers of Sequence ID NOs. 5-12 according to the procedure disclosed in Reference Example 2.

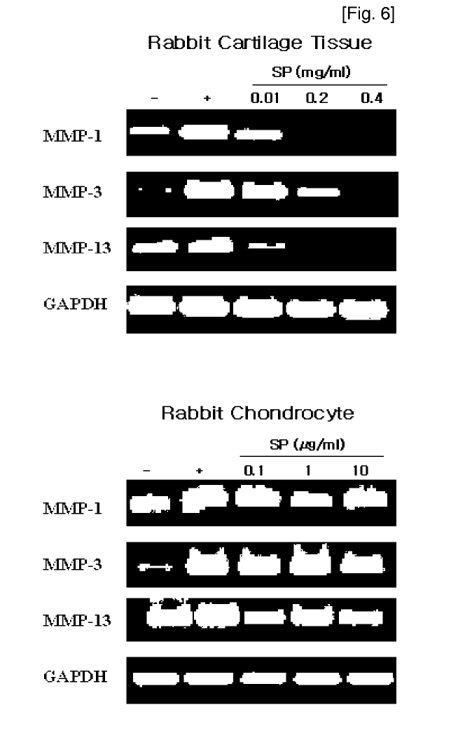

As shown in FIG. 6, the inventive extract of the present invention significantly inhibited the gene expression of MMP-1, MMP-3 and MMP-13, particularly, MMP-1 in cartilage cell in a dose dependent manner. The inhibition of MMP-3 and MMP-13 was weaker than that of MMP-1 however the inhibition was dose dependent.

1-7. Cell Toxicity Test

In order to examine the effect of inventive extract on the viability of chondrocyte, the cell toxicity test using the cartilage tissue and chondrocyte of rabbit prepared in Reference Example 1-1 and 1-2 was performed according to the method disclosed in the literature (Cakmak O et al., Arch Facial Plast. Surg., 7(6), pp 406-409, 2005).

As an indicative of chondrocyte viability, the activity of cytoplasmic enzyme lactate dehydrogenase (LDH) was measured by conventionally available kit (LDH kit, Promega Corp., Madison, Wis., USA) and MTT (methylthiazolyldiphenyl-tetrazolium bromide) assay was performed according to the procedure disclosed in the literature (Hussain S M et al., Toxicol. In Vitro, 19(7) pp 975-983, 2005).

To determine the activity of LDH, the negative control group and test group treated with inventive extract was incubated for 3 days to collect the culture medium. After dissolving the substrate mixed powder (diaphrase, lactate, NAD) in TBT solution (Tris-buffered Tetrazolium), 50□ of medium was mixed with 50 [symbol] of substrate mixture to react together at room temperature for 30 minutes. After adding 50 [symbol] of stopping solution thereto, the absorbance of culture medium was measured at 490 nm to determine the activity of LDH.

To perform MTT assay, the chondrocyte was added to 96-well plates and varius concentrations of inventive extract (0.01, 0.1, 1, 10, 100 [symbol] ) was added thereto to incubate for 72 hours. After adding WST-8 (water-soluble tetrazolium salt) solution thereto, the medium was incubated for about 2 hours and the absorbance was determined at 450 nm when the WST-8 formazan was formed.

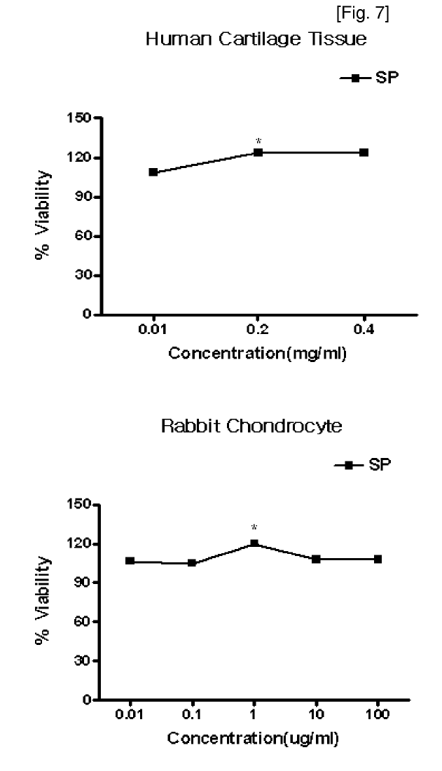

As shown in FIG. 7, both of 0.4 [symbol] and 100 [symbol] of inventive extract did not affect on the viability of cultured human cartilage tissue for 3 days. Accordingly, it has been confirmed that the inventive extract of the present invention did not show cell toxicity in cartilage tissue and chondrocyte and safe.

1-8. Recovery of Cartilage Tissue

In order to confirm the effect on the recovery of cartilage tissue or Chondrocyte, following experiment was performed according to the method disclosed in the literature (Byron C R et al., Am. J. Vet. Res., 66(10), pp 1757-1763).

The cultured slices of the cartilage tissue of human and rabbit prepared in Reference Example 1-1 was fixed in 10% neutral formalin, dehydrated with ethanol and embedded with paraffin.

The paraffin block was sectionalized to the thickness of 4 [symbol] and attached to poly-L-lysine-coated glass slide (Sigma, USA). The slices was subjected to deparaffinization, hydration process and staining with hematoxylin and eosin.

In order to stain each proteoglycan and collagen in cartilage tissue, the slices was stained with safranin O (Sigma, USA) and trichrome (Sigma, USA)(Muir H M et al., Histology, Churchill Livingstone, Edinburgh, pp 177-198, 1986).

The pathologist who had not recognized the information on the sample was interpreted the stained slides and the slide was photographed with the lens (200×).

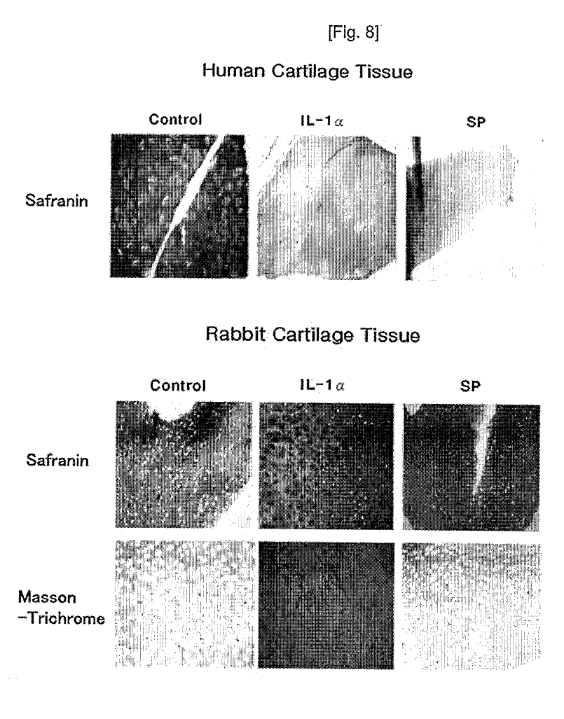

As shown in FIG. 8, the extent of staining of human cartilage tissue was weaker and the thickness of cartilage was thinner than those of normal cartilage tissue since the loss of proteoglycan and collagen in inflamed cartilage cell with the induction of IL-1α. However, the inventive extract recovered the loss of cartilage tissue induced by interlukin-1α, which confirmed the increased concentration of proteoglycan stained with safranin O. In rabbit cartilage cell, the stained proteoglycan and collagen with each safranin O and trichrome in test group treated with inventive extract was darken, which confirmed that the inventive extract recovered the loss of cartilage tissue by induced interlukin-1α and did not show any morphological change compared with that of normal group.

### Experimental Example 2

### Analgesic and Antiphlogistic Activity

2-1. Acetic Acid Induced Writhing Analgesia Test

In order to determine the analgesic activity of the inventive extract of the present invention, acetic acid-induced writhing test was performed according to the method disclosed in the literature as follows (Whittle B A, Brit. J. Pharmacol. Chemother., 22, pp 246-253, 1964).

Male ICR mouse (Jung Ang laboratory animal, Japan) weighing from 20 to 25 g was acclimated for several days and each group consists of 10 mice. The extract of Siegesbeckiae herba (500 mg/kg) and COX-2 inhibitor celecoxib (100 mg/kg, Sigma, USA) used as a positive control were administrated orally into the mice and 0.7% acetic acid (v/v in DW) was abdominally administrated to induce pain one hour after the administration. From 5 minutes later, the writhing frequency of mice was observed for 10 minutes and the inhibition rate (%) was calculated based on that of negative control.

TABLE 2

| | Concentration | Inhibition rate |
|---|---|---|
| sample | (mg/kg) | of edema (%) |
| The extract of | 500 | 71.1 |
| Siegesbeckiae herba (SP) | | |
| Celecoxib | 100 | 61.9 |

As shown in Table 2, the inventive extract (SP) showed potent inhibitory effect on the pain. Accordingly, it has been confirmed that the extract of Siegesbeckiae herba showed more potent inhibitory effect than Celecoxib, a conventionally used NSAID.

2-2. Test with Arachidonic Acid Induced Ear Edema

Arachidonic acid is a precursor of prostaglandin inducing various inflammatory diseases, such as blood platelet coagulation, coronary sclerosis, heart disease, inflammation and so on.

In order to determine the anti-inflammatory activity of inventive extract, following test using arachidonic acid-induced ear edema was performed according to method disclosed in the literature (Zhou H et al., Biol. Pharm. Bull., 29(2) pp 253-260, 2006).

Male ICR mouse (Jung Ang laboratory animal, Japan) weighing from 20 to 30 g was used as an experimental animal and each group consists of 10 mice. The extract of Siegesbeckiae herba (SP, 500 mg/kg) and Celecoxib, a COX-2 inhibitor (100 mg/kg, Sigma, USA) used as a positive control were administrated orally and after 1 hours, 50 mg/ml of arachidonic acid dissolved in acetone was spread on the inner and outer surface of right ear to induce ear-edema. After 1 hour, the increased rate of ear thickness was calculated by comparing with that of left ear and the result was shown in Table 3.

TABLE 3

| | Concentration | Inhibition rate |
|---|---|---|
| Sample | (mg/kg) | of edema (%) |
| Extract of Siegesbeckiae | 500 | 21.04 |
| herba (SP) | | |
| Celecoxib | 100 | 25.29 |

As can be seen in Table 3, the inventive extract (SP) showed potent anti-inflammatory effect on the ear-edema. Accordingly, it has been confirmed that the extract of Siegesbeckiae herba showed similar anti-inflammatory effect to Celecoxib, a conventionally used NSAID.

2-3. Test with Croton Oil Induced Ear Oedema

Croton oil induces various skin inflammations such as rash, swelling, blister and so on.

In order to determine the anti-inflammatory activity of inventive extract, following test using croton oil-induced ear edema was performed according to method disclosed in the literature (Zhang B et al., Eur. J. Pharmacol., 530(1-2), pp 166-171, 2006).

Male ICR mouse (Jung Ang laboratory animal, Japan) weighing from 20 to 30 g was used as an experimental animal and each group consists of 6 mice. The extract of Siegesbeckiae herba (SP, 500 mg/kg) and Celecoxib, a COX-2 inhibitor (100 mg/kg, Sigma, USA) used as a positive control were administrated orally and after 1 hours, 2.5% croton oil dissolved in acetone was spread on the inner and outer surface of right ear to induce ear-edema. After 4 hours, the increased rate of ear thickness was calculated by comparing with that of left ear and the result was shown in Table 4.

TABLE 4

| | Concentration | Inhibition rate |
|---|---|---|
| Sample | (mg/kg) | of edema (%) |
| Extract of Siegesbeckiae | 500 | 31.18 |
| herba (SP) | | |
| Celecoxib | 100 | 33.12 |

As can be seen in Table 4, the inventive extract (SP) showed potent anti-inflammatory effect on the ear-edema. Accordingly, it has been confirmed that the extract of Siegesbeckiae herba showed similar anti-inflammatory effect to Celecoxib, a conventionally used NSAID.

2-4. Inhibition Test of Prostaglandin Activity

In order to confirm the inhibitory effect of inventive extract on PGE2 activity, the inhibition test of prostaglandin activity was performed according to the method disclosed in the literature (Zhou H et al., Biol. Pharm. Bull., 29(2) pp 253-260, 2006).

Diluted RAW 264.7 cell (American Type Culture Collection, Manassas, Va.) to the concentration of 5×10⁵ cell/ [symbol] was add to 6-well plate containing inactivated fetal bovine serum (FBS, GIBCO BRL, USA) with heat and 100 unit/ [symbol] of penicillinstreptomycin and each 200 [symbol] of each cell was added to 96-well plate to incubate for 24 hours. 4 hours before the treatment of the sample, 500 μm of aspirin was added thereto to inhibit the activity and expression of COX-1 in the cell. The activity of PGE2 due to activity of sole COX-2 was determined. The inventive extract of the present invention was treated therewith in a various concentration and incubated for 24 hours. The inhibitory effect of each extract on inflammation was determined by measuring the amount of PGE2 according to ELISA method (Endogen, Cambridge, Mass.) and the inhibition rate (%) was calculated by setting the inhibition rate in negative group to zero.

TABLE 5

| | | |
|---|---|---|
| | Concentration | Inhibition |
| Experimental group | of treatment | rate (%) |

| | | | |
|---|---|---|---|
| Negative group | 0 | | 0 |
| (Non-treatment group) | | | |
| Extract of Siegesbeckiae | 30 | □/□ | 14.5 |
| herba (SP) | 100 | □/□ | 25.6 |
| | 300 | □/□ | 47.5 |
| Diclofenac | 30 | μM | 12.4 |
| Celecoxib | 30 | μM | 67.5 |

As shown in Table 5, the inventive extract of the present invention inhibited the expression of PGE2 in a dose dependent manner while diclofenac, a non-selective COX-2 inhibitor, nearly inhibited the expression. Celecoxib, a selective inhibitor, showed potently inhibit the expression. Accordingly, the inventive extract of the present invention inhibited the activity of PGE2 similar to Celecoxib as a selective COX-2 inhibitor.

2-5. Inhibition of TNF-α

TNF-α is an important inflammatory cytokines inducing to initial inflammatory reaction against the immuno-reaction of infectious microorganism as well as an important indicative of arthritis since it plays an important role in stimulating the inflammation of articular tissue in arthritis and rheumatic arthritis.

In order to confirm the inhibition effect of inventive extract on TNF-α production, following experiment was performed according to the method disclosed in the literature (Lin R et al., Curr. Neurovasc. Res., 3(1), pp 41-47, 2006).

Diluted RAW 264.7 cell (American Type Culture Collection, Manassas, Va.) to the concentration of 2×10⁴ cell/ [symbol] was add to 6-well plate containing inactivated fetal bovine serum (FBS, GIBCO BRL, USA) with heat and 100 unit/ [symbol] of penicillinstreptomycin.

1 mg/ml of LPS was added to the cell to induce inflammation and then various concentrations of inventive extract were added thereto to incubate for 24 hours. The inhibition effect on inflammation was determined by measuring the concentration of TNF-α in medium according to ELISA method (Endogen, Cambridge, Mass.).

TABLE 6

| Sample | concentration of SP(□/ml) | TNF-α(pg/ml) |
|---|---|---|
| LPS treatment + SP | 10 | 3.43 |
| | 50 | 2.15 |
| | 200 | 1.02 |
| Non-treatment | — | 0.85 |
| LPS-treatment | — | 3.67 |

As shown in Table 6, the inventive extract of the present invention inhibited the TNF-α production induced by LPS in a dose dependent manner, which confirmed that the extract of Siegesbeckiae herba showed potent inhibitory effect on inflammation.

2-6. Inhibition of No (Nitric Oxide) Production (In Vitro)

In order to confirm the inhibitory effect of the inventive extract on NO activity, following experiment was performed according to the method disclosed in the literature (Lin R et al., Curr. Neurovasc. Res., 3(1), pp 41-47, 2006).

Nitrite accumulation, an indicative of NO synthesis, was measured by applying Griess reaction. Peritoneal macrophage was incubated in RPMI (GIBCO BRL, USA) medium containing inactivated fetal bovine serum (FBS, GIBCO BRL, USA) with heat, 100 unit/ [symbol] of penicillin and 100 unit/ [symbol] of streptomycin sulfate, and incubated at 37° C. in 5% CO_{z} incubator. 1, 10, 100, 300 [symbol] of each extract of Siegesbeckiae herba was added to 96-well plates with 1 [symbol] of LPS, 1 ng/ [symbol] of IFN-gamma to be the concentration of 1×10⁵ cell/well. After incubating for 3 days, 100 [symbol] of collected cell culture medium was mixed with 5% (v/v) griess reagent, 100□ of 1% (w/v) sulfanilamide dissolved in equivalent amount of phosphoric acid and naphthyl ethylene diamine-hydrochloric acid, and incubated at room temperature for 10 minutes. The absorbance was measured at 550 nm by using micro plate reader (Power Wave 340, Bio-Tek, USA). Fresh medium in all experiments was used as non-treatment group. The amount of NO in the medium was calculated based on the generated sodium nitrite standard curve and result was showed in Table 7.

TABLE 7

| Sample | Concentration (□/mL) | NO (□/ml) |
|---|---|---|
| SP treatment | 1 | 47.32 |
| | 10 | 21.33 |
| | 100 | 10.51 |
| | 300 | 4.41 |
| LPS treatment | — | 51.97 |
| Non-treatment | — | 1.61 |

As shown in Table 7, the amount of produced NO induced by LPS was about 32.5 times as much as that of Non-treatment group, which confirmed that the extract of Siegesbeckiae herba (SP) inhibited the NO production in a dose dependent manner.

### Experimental Example 3

### Acute Toxicity Test of Oral Administration in Rat

The acute toxicity test was performed by administrating inventive extract to 6-weeks aged SPF Sprague-Dawley rats.

250 mg/kg, 500 mg/kg, 1000 mg/kg, 5000 mg/kg of inventive extract was orally administrated to each group consisting of 2 rats and the symptoms of rats were observed for 14 days. After administrating the extract, all the clinical changes i.e., mortality, clinical signs, body weight changes was observed and blood test such as haematological test and hematological biochemistry test was performed. The abnormal changes of abdominal organ and thoracic organ were observed after autopsy.

There did not show any changes in mortality, clinical signs, body weight changes and gross findings in any group or either gender. Furthermore, there showed any toxicity in test group treated with 5000 mg/kg of inventive extract.

Accordingly, it has been confirmed that the inventive extract prepared in the present invention was potent and safe substance showing LD₅₀ (more than 5000 mg/kg) in oral administration.

Hereinafter, the formulating methods and kinds of excipients will be described, but the present invention is not limited to them. The representative preparation examples were described as follows.

Preparation of Powder

SP extract 20 mg

Lactose 100 mg

Talc 10 mg

Powder preparation was prepared by mixing above components and filling sealed package.

Preparation of Tablet

SP extract 10 mg

Corn Starch 100 mg

Lactose 100 mg

Magnesium Stearate 2 mg

Tablet preparation was prepared by mixing above components and entabletting.

Preparation of Capsule

SP extract 10 mg

Corn Starch 3 mg

Lactose 14.8 mg

Magnesium Stearate 0.2 mg

Capsule preparation was prepared by mixing above components and filling gelatin capsule by conventional gelatin preparation method.

Preparation of Injection

SP extract 10 mg

Mannitol 180 mg

Distilled water for injection 2974 mg

Na₂HPO₄, 12H₂O 26 mg

Injection preparation was prepared by dissolving the components in 2 [symbol] ample and sterilizing by conventional injection preparation method.

Preparation of Health Care Food

SP extract 1000 mg

Vitamin mixture optimum amount

Vitamin A acetate 70 mg

Vitamin E 1.0 mg

Vitamin B₁ 0.13 mg

Vitamin B₂ 0.15 mg

Vitamin B₁ 0.5 mg

Vitamin B₂ 0.2 mg

Vitamin C 10 mg

Biotin 10 mg

Amide nicotinic acid 1.7 mg

Folic acid 50 mg

Calcium pantothenic acid 0.5 mg

Mineral mixture optimum amount

Ferrous sulfate 1.75 mg

Zinc oxide 0.82 mg

Magnesium carbonate 25.3 mg

Monopotassium phosphate 15 mg

Dicalcium phosphate 55 mg

Potassium citrate 90 mg

Calcium carbonate 100 mg

Magnesium chloride 24.8 mg

The above mentioned vitamin and mineral mixture may be varied in may ways.

Such variations are not to be regarded as a departure from the spirit and scope of the present invention.

Preparation of Health Beverage

SP extract 1000 mg

Citric acid 1000 mg

Oligosaccharide 100 g

Apricot concentration 2 g

Taurine 1 g

Concentrated plum solution 2 g

Distilled water 900

[symbol]

Health beverage preparation was prepared by dissolving active component, mixing, stirred at 85° C. for 1 hour, filtered and then filling all the components in 2000□ ample and sterilizing by conventional health beverage preparation method.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

### INDUSTRIAL APPLICABILITY

As described in the above, the extract of Siegesbeckiae herba showed inhibitory effect on the dissociation of proteoglycan and type II collagen in chondrocyte and cartilage tissue and protecting effect on cartilage due to the inhibition of MMP-1, MMP-3 and MMP-13 activity and the restoring effect on cartilage tissue, the anti-inflammatory and antiphlogistic effect in edema animal model, anti-inflammatory effect confirmed by the inhibition test on PGE2 activity through COX-2 inhibition and the inhibition test of the reproduction of TNF-α and NO, it can be used as the therapeutics or health food for treating and preventing arthritis.

1. A pharmaceutical composition comprising an extract of Siegesbeckiae Herba and a pharmaceutically acceptable carrier thereof as an active ingredient for treating and preventing arthritic disease.

2. The pharmaceutical composition of claim 1, wherein said extract is extracted with the solvent selected from the group consisting of water, C₁ to C lower alcohol and the mixture thereof.

3. The pharmaceutical composition of claim 1, wherein said arthritic disease is degenerative arthritis or rheumatic arthritis.

4. A use of an extract of Siegesbeckiae Herba for the preparation of therapeutic agent for the treatment and prevention of arthritic disease in human or mammal.

5. A health care food comprising an extract of Siegesbeckiae Herba, together with a sitologically acceptable additive for prevention and alleviation of arthritic disease.

6. The health food according to claim 5 wherein said health food is provided as powder, granule, tablet, capsule or beverage type.


SEQUENCE LISTING (12 sequences)

SEQ ID NO: 1 - DNA, 20 bp, Artificial Sequence
  Col II forward primer
aacactgccaacgtccagat

SEQ ID NO: 2 - DNA, 20 bp, Artificial Sequence
  Col II reverse primer
ctgcagcacggtataggtga

SEQ ID NO: 3 - DNA, 20 bp, Artificial Sequence
  PG forward primer
gaggtcgtggtgaaaggtgt

SEQ ID NO: 4 - DNA, 20 bp, Artificial Sequence
  PG reverse primer
gtgtggatggggtacctgac

SEQ ID NO: 5 - DNA, 19 bp, Artificial Sequence
  MMP-1 forward primer
aaagggaataagtactggg

SEQ ID NO: 6 - DNA, 22 bp, Artificial Sequence
  MMP-1 reverse primer
gtttttccagtgttttcctcag

SEQ ID NO: 7 - DNA, 21 bp, Artificial Sequence
  MMP-3 forward primer
tgcgtggcagtttgctcagcc

SEQ ID NO: 8 - DNA, 21 bp, Artificial Sequence
  MMP-3 reverse primer
gaatgtgagtggagtcacctc

SEQ ID NO: 9 - DNA, 19 bp, Artificial Sequence
  MMP-13 forward primer
gataaagactatccgagac

SEQ ID NO: 10 - DNA, 18 bp, Artificial Sequence
  MMP-13 reverse primer
cgaacaatacggttactc

SEQ ID NO: 11 - DNA, 24 bp, Artificial Sequence
  GAPDH forward primer
gctctccagaacatcatccctgcc

SEQ ID NO: 12 - DNA, 24 bp, Artificial Sequence
  GAPDH reverse primer
cgttgtcataccaggaaatgagct